(19) United States
(12) Patent Application Publication
(10) Pub. No.: US 2025/0050120 A1
(43) Pub. Date: Feb. 13, 2025

(54) AUTOMATED EXTERNAL DEFIBRILLATOR

(71) Applicant: CELLAED LIFE SAVER PTY. LTD., Sydney, NSW (AU)

(72) Inventors: Donovan Lachlan CASEY, Sydney, New South Wales (AU); Erol Erdogan TEBER, Sydney, New South Wales (AU)

(21) Appl. No.: 18/723,136

(22) PCT Filed: Dec. 23, 2022
(86) PCT No.: PCT/IB2022/062752

§ 371 Date: Jun. 21, 2024

(30) Foreign Application Priority Data
Dec. 23, 2021 (AU) 2021904260

(51) Int. Cl. A61N 1/39

(52) U.S. Cl. CPC A61N 1/3904

(57) ABSTRACT

Described is an automated external defibrillator (AED). The AED comprises two pads for placement on a patient, each pad comprising an energy storage system. The energy storage system comprises at least two energy storage blocks, a switching circuit and a shock generation circuit connected to the two pads, and a controller connected to the switching circuit and the shock generation circuit. The controller is configured to perform an electrical switching operation to provide a defibrillation shock in two phases, such that the voltage and a peak current in each of the two phases is substantially the same. Each energy storage block comprises one or more capacitors. At least one of the energy storage blocks including two or more capacitors connected in series, and at least two energy storage blocks are connected in parallel so that the capacitor system includes capacitors connected both in series and in parallel with each other.

### FIELD OF TECHNOLOGY

The present invention relates to automated external defibrillators having a small form factor yet capable of providing a defibrillation shock in two phases, such that the voltage and a peak current in each of the two phases is substantially the same.

The present invention further relates to automated external defibrillators having energy storage systems comprising capacitors connected both in series and in parallel.

### BACKGROUND

The automated external defibrillators (AED) automatically diagnoses the presence of a shockable arrhythmia and delivers an electrical shock to restore the heart rhythm to normal.

The majority of AEDs available in the market are large devices that are typically stored in wall cabinets and are costly. These AEDs have a large form factor to house the necessary electrical componentry to operate the AED which delivers a large volt charge in a fraction of a second. For the most part, such AEDs are few and far in between and in an emergency difficult to locate and use quickly. Thus AEDs continue to be unavailable to victims or bystanders in the majority of cases of sudden cardiac arrest.

Conversely, an implantable cardioverter-defibrillator (ICD) is a small battery-powered device placed in the chest to detect and stop irregular heartbeats (arrhythmias). An ICD continuously monitors the heartbeat and delivers electric shocks, when needed, to restore a regular heart rhythm. An ICD delivers a lower voltage to the heart compared to an AED since it connects directly to the heart compared to the resistance provided by the skin and tissue above the heart when using an AED.

It is an object of the disclosure provide an improved energy storage system and artificial external defibrillator having the same which addresses or ameliorates one or more disadvantages or limitations associated with the prior art, or at least which provides the public with a useful choice.

### SUMMARY

In a first aspect, the disclosure may provide an automated external defibrillator (AED) comprising
two pads for placement on a patient, each pad comprising an energy storage system, the energy storage system comprising at least two energy storage blocks,
a switching circuit and a shock generation circuit connected to the two pads, and
a controller connected to the switching circuit and the shock generation circuit, the controller configured to perform an electrical switching operation to provide a defibrillation shock in two phases, such that the voltage and a peak current in each of the two phases is substantially the same.

In another aspect, the disclosure may provide a method of operating an AED having two pads for placement on a patient, the method comprising:
performing multiple functions of electrical measurement and stimulation of the patient's heart, and
operating a controller to perform an electrical switching operation to provide a defibrillation shock in two phases, wherein a voltage and a peak current in each of the two phases is substantially the same.

In another aspect, the disclosure may provide a defibrillator comprising
two pads for placement on a patient,
a capacitor system having at least two energy storage blocks, each energy storage block comprising a capacitor, and the at least two energy storage blocks being connected in parallel,
a switching circuit and a shock generation circuit connected to the two pads, and
a controller connected to the switching circuit and the shock generation circuit, the controller configured to perform an electrical switching operation to provide a defibrillation shock in two phases, such that the voltage and a peak current in each of the two phases is substantially the same.

In another aspect, the disclosure may provide a defibrillator comprising a capacitor system having at least two energy storage blocks, each energy storage block comprising a capacitor, and at least one of the energy storage blocks comprising two or more capacitors connected in series, and at least two energy storage blocks are connected in parallel so that the capacitor system includes capacitors connected both in series and in parallel with each other, and wherein the series and parallel arrangement of the capacitors of the capacitor system are the same during both a charging of the capacitor system and a discharging of the energy system to provide a defibrillation shock.

In another aspect, the disclosure provides a defibrillator comprising a capacitor system having a plurality of energy storage blocks fixed in parallel with each other and each energy storage block includes a capacitor and at least one of the energy storage blocks comprising two or more capacitors fixed in series.

In another aspect, the disclosure provides for a defibrillator comprising an energy storage system having at least two energy storage blocks electrically connected in parallel, wherein each of the energy storage blocks includes a capacitor and at least one of the energy storage blocks includes two or more capacitors electrically connected in series such that the energy storage system includes capacitors arranged in both series and parallel, and wherein the series and parallel arrangements of the capacitors of the energy storage system are the same during both a charging of the energy system and a discharging of the energy system to provide a defibrillation shock.

In another aspect, the disclosure provides for a defibrillator comprising an energy storage system having at least two capacitor blocks electrically connected in parallel, wherein at least one of the capacitor blocks comprises two or more capacitors electrically connected in series, and wherein the series and parallel arrangement of the capacitors of the energy storage system are the same during both a charging of the energy system and a discharging of the energy system to provide a defibrillation shock.

In another aspect, the disclosure provides for an energy storage system for a defibrillator, the energy storage system associated with a battery to charge it and a pair of electrode pads through which the stored energy can be delivered as a defibrillation shock, the energy storage system comprising a plurality of energy storage blocks connected together in parallel, wherein each energy storage block includes a plurality of capacitors connected together in series.

In another aspect, the disclosure provides for a charge storage assembly for an automatic external defibrillator, the charge storage assembly comprising:
a plurality of charge storage blocks fixedly connected in parallel,
wherein each charge storage block comprises a plurality of capacitors fixedly connected in series.

In another aspect, the disclosure provides for an artificial external defibrillator with a first and second banks of capacitors, wherein at least one bank includes at least three capacitors, where at least one capacitor is connected in series to another of the capacitors, and at least one capacitor is connected in parallel to another of the capacitors, and wherein the series and parallel arrangement of the capacitors are fixed.

In another aspect, the disclosure provides for a method of operating an automatic external defibrillator, the method comprising the steps of:
charging a capacitor system from a battery, the capacitor system comprising a first and second capacitor banks each comprising at least three capacitors, where at least one capacitor of each capacitor bank is connected in series to another of the capacitors of the same capacitor bank, and at least one capacitor of each capacitor bank is connected in parallel to at least another of the capacitors of the same capacitor bank, and
discharging the first capacitor bank to provide a first defibrillation phase, and
discharging the second capacitor bank to provide a second defibrillation phase, and
wherein the series and parallel arrangement of the capacitors of the first and second capacitor banks are the same for both the charging step and each of the discharging steps.

In another aspect, the disclosure provides for a method of charging an automatic external defibrillator, the method comprising the step of:
charging a capacitor system from a battery, where the capacitor system comprises at least two capacitors fixedly connected in series and at least one capacitor fixedly connected in parallel to the at least two capacitors fixedly connected in series.

In another aspect, the disclosure provides for a method of manufacturing a capacitor system for an automatic external defibrillator, the method comprising:
fixedly connecting a plurality of capacitors in series to form an energy storage block,
providing a plurality of said energy storage blocks, and
fixedly connecting the plurality of energy storage blocks together in parallel to form the capacitor system.

In another aspect, the disclosure provides for a defibrillator comprising a plurality of capacitors to store energy for defibrillation, wherein the capacitors are cylindrical and have a longitudinal axis, and wherein the capacitors are arranged within the defibrillator so the longitudinal axis of each capacitor are located within a common plane.

The following configurations may relate to any of the above aspects.

In one configuration the at least two energy storage blocks are independent of each other for each of the two phases of the defibrillation shock.

In one configuration the at least two energy storage blocks are connected in parallel, each energy storage block comprising at least one capacitor and at least one of the energy storage blocks comprising two or more capacitors in series.

In one configuration the series and parallel arrangement of the capacitors are the same during both charging of the energy storage blocks and discharging of the energy storage blocks, to provide a defibrillation shock.

In one configuration each pad has a volume of about 100 cm³ to 200 cm³, and a surface area of about 50 cm² to 100 cm², and suitable ranges may be selected from between any of these values.

In one configuration the controller is further configured to produce an equal leading edge waveform for each of the two phases.

In one configuration the controller is further configured to generate a predetermined dosage of current for defibrillation shock at a predetermined dosage of power.

In one configuration the controller is further configured to maintain peak current in each phase such that the polarisation effect is observed in the first phase and a depolarisation effect is achieved in the second phase.

In one configuration the controller is further configured to produce an fully tilted waveform for each of the two phases.

In one configuration the switching circuit is configured to perform electrical switching operation such that one of the energy storage blocks is configured to charge, store and discharge to provide energy for one of the two phases, and the other of the energy storage blocks is configured to charge, store and discharge to provide energy for the other of the two phases of the defibrillation shock.

In one configuration the switching circuit is configured to perform electrical switching operation such that the direction of the current flow is maintained during each of the two phases during the defibrillation shock.

In one configuration each of the capacitors of at least one of the energy storage blocks have the same or substantially the same nominal capacitance and working voltage.

In one configuration each energy storage block further comprises any one or more of a balancing resistor, a diode, or an operational amplifier connected in series and/or parallel connection with the at least one capacitor in each of the energy storage blocks.

In one configuration the AED further comprises any one or more of a transformer, electrical switch, battery and an inductor, and wherein, each of the transformer, electrical switch, battery and the inductor are configured to be operable in a low voltage or a low power mode.

In one configuration the shock generation circuit comprises a charging circuit and/or a discharging circuit configured to charge and/or discharge the one or more capacitors of the energy storage blocks.

In one configuration the controller is configured to operate the shock generation and switching circuit to automatically perform electrical measurement and stimulation of the patient's heart switching between the two phases.

In one configuration each of the two pads comprises one or more electrodes, and wherein the at least one electrode of each pads is configured to carry out at least one of an electrical measurement and stimulation of the patient's heart.

In one configuration the peak current and voltage in the first of the two phases of the defibrillation shock are maintained until a first time interval tp1 in which a polarisation effect is observed in the patient,

In one configuration the first time interval is the time taken for the defibrillation shock to reach all cells of myocardium of the patient.

In one configuration the multiple functions of electrical measurement and stimulation of the patient's heart performed by the one or more electrodes in multiple directions comprise:
measuring cardiac electrical signals to detect locations of the two pads;
measuring ECG signals to detect shockable cardiac rhythms; and
delivering doses of defibrillation shocks by the two pads based on their detected locations when shockable cardiac rhythms are detected.

In one configuration the measured cardiac electrical signals used to detect locations of the two pads comprise voltage, current, impedance, or any combination thereof.

In one configuration the capacitors of each energy storage block are fixed in series.

In one configuration the series and parallel arrangement of the capacitors of the energy storage system is non-switchable.

In one configuration the electrical connections between the capacitors of the capacitor system are unswitched.

In one configuration the capacitors of the energy storage system have fixed unswitched connections between them.

In one configuration each of the energy storage blocks have at least two capacitors connected in series.

In one configuration each of the energy storage blocks have an equal plurality of capacitors connected in series.

In one configuration the first capacitor bank and second capacitor bank each have the same total working voltage.

In one configuration each of the capacitors of at least one of the energy storage blocks have the same or substantially the same nominal capacitance and working voltage.

In one configuration each of the capacitors of each of the energy storage blocks have the same or substantially the same nominal capacitance and working voltage.

In one configuration each of the capacitors of at least one of the energy storage blocks are identical.

In one configuration each of the capacitors of each of the energy storage blocks are identical.

In one configuration each energy storage block comprises a balancing resistor connected in parallel with the, or each, capacitor of each respective energy storage block.

In one configuration the balancing resistor or resistors are configured to maintain a uniform voltage across each capacitor of each respective energy storage block.

In one configuration the capacitors of the capacitor system are of a cylindrical form and have a longitudinal axis, and the capacitors are arranged such that each of the longitudinal axes lie in a plane.

In one configuration the capacitors of the capacitor system are of a cylindrical form and have a longitudinal axis, and the capacitors are arranged such that the longitudinal axis of each the capacitors are either a) parallel or b) co-axial with the longitudinal axis of another of the capacitors.

In one configuration the capacitor system comprises a first capacitor bank and a second capacitor bank, wherein each capacitor bank includes at least two energy storage blocks, and at least one of the energy storage blocks of each capacitor bank includes two or more capacitors connected in series.

In one configuration the first capacitor bank and second capacitor bank are each configured to store charge and discharge to provide energy, respectively, for a first phase and a second phase of a biphasic defibrillation shock.

In one configuration the defibrillator comprises four discharge switches, each switch being between each of the first and second capacitor banks.

In one configuration the first capacitor bank supplies charge for a first phase of a biphasic defibrillation shock and the second capacitor bank supplies charge for a second phase of a biphasic defibrillation shock.

In one configuration charge is not transferred between the first capacitor bank and the second capacitor bank.

In one configuration the first capacitor bank and second capacitor bank have different total capacitances.

In one configuration the first capacitor bank has about half again the total capacitance of the second capacitor bank.

In one configuration the first capacitor bank includes a greater number of energy storage blocks than the second capacitor bank.

In one configuration each of the energy storage blocks include four capacitors connected in series.

In one configuration the second capacitor bank comprises four energy storage blocks connected in parallel.

In one configuration each of the energy storage blocks include four capacitors connected in series.

In one configuration a capacitor of an energy storage block has a nominal capacitance of about 6.8 μF.

In one configuration a capacitor of each energy storage block of one or both of the first capacitor bank and second capacitor bank has a nominal capacitance of about 6.8 μF.

In one configuration each capacitor of each energy storage block of one or both of the first capacitor bank and second capacitor bank has a nominal capacitance of about 6.8 μF.

In one configuration a capacitor of an energy storage block has a working voltage of about 450 V.

In one configuration a capacitor of each energy storage block of one or both of the first capacitor bank and second capacitor bank has a working voltage of about 450 V.

In one configuration each capacitor of each energy storage block of one or both of the first capacitor bank and second capacitor bank has a working voltage of about 450 V.

In one configuration the defibrillator further comprises a battery to charge the capacitor system and a pair of electrode pads to deliver the stored energy to a patient as the defibrillation shock.

In one configuration the defibrillator further comprises a charging circuit configured to charge the capacitor system from the battery.

In one configuration the third capacitor is connected in parallel with both of the first capacitor and second capacitor.

In one configuration each of the capacitors of at least one of the energy storage blocks have the same or substantially the same nominal capacitance and working voltage.

In one configuration the plurality of charge storage blocks are arranged in a first and second charge storage banks, wherein the banks are for isolated and successive discharge.

In one configuration during each of the discharge steps, the capacitors of the first and second capacitor banks are electrically isolated from each other.

In one configuration the number of capacitors of each energy storage block are selected based on a working voltage of the capacitors and a desired working voltage of the capacitor system.

In one configuration the number energy storage blocks are selected based on a nominal capacitance of the capacitors and a desired total nominal capacitance of the capacitor system.

In one configuration at least some of the capacitors are aligned axially parallel to one or more of the others of the capacitors.

In one configuration each of the capacitors are aligned axially parallel with one or more of the others of the capacitors.

The term “axis” as used in this specification means the axis of revolution about which a line or a plane may be revolved to form a symmetrical shape. For example, a line revolved around an axis of revolution will form a surface, while a plane revolved around an axis of revolution will form a solid.

As used herein the term “and/or” means “and” or “or”, or both.

As used herein “(s)” following a noun means the plural and/or singular forms of the noun.

For the purpose of this specification, where method steps are described in sequence, the sequence does not necessarily mean that the steps are to be chronologically ordered in that sequence, unless there is no other logical manner of interpreting the sequence.

The term “comprising” as used in the specification and claims means “consisting at least in part of.” When interpreting each statement in this specification that includes the term “comprising,” features other than that or those prefaced by the term may also be present. Related terms “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other aspects of the invention may become apparent from the following description which is given by way of example only and with reference to the accompanying drawings.

### BRIEF DESCRIPTION OF THE DRAWINGS

Preferred embodiments of the invention will be described by way of example only and with reference to the drawings, in which:

FIG. 1 is a circuit diagram of a capacitor system or part thereof for a defibrillator, illustrating its two energy storage blocks.

FIG. 2 is a simplified circuit diagram of a defibrillator with a capacitor storage system.

FIG. 3 is a circuit diagram of a capacitor storage system or part thereof.

FIG. 4 is a partial view of a simplified circuit diagram of a defibrillator with a capacitor storage system.

FIG. 5 is a simplified circuit diagram of a defibrillator with a capacitor storage system having two banks of capacitors.

FIG. 6A is a circuit diagram of a capacitor bank of capacitor system for a defibrillator.

FIG. 6B is a view of the circuit diagram of FIG. 9A, illustrating the energy storage blocks of the capacitor bank.

FIG. 7 is a partial view of a simplified circuit diagram of a defibrillator with a capacitor storage system having two banks of capacitors.

FIG. 8 is a partial view of a simplified circuit diagram of a defibrillator with a capacitor system having two banks of capacitors.

FIG. 9A is a view of a defibrillator or part of a defibrillator, illustrating an internal component.

FIG. 9B is a view of a defibrillator or part of a defibrillator, illustrating various internal components.

FIG. 10A is another view of a defibrillator or part of a defibrillator, illustrating various internal components.

FIG. 10B is another view of a defibrillator or part of a defibrillator, illustrating various internal components.

FIG. 10C is another view of a defibrillator or part of a defibrillator, illustrating various internal components.

FIG. 11 is an illustration of a defibrillator provided on a patient's torso.

FIG. 12 is a view of a defibrillator.

FIGS. 13A-C are schematics illustrating arrangements of a capacitor system within a defibrillator.

FIG. 14 is an exemplary embodiment of the capacitor configurations for each energy storage bank.

FIG. 15 is an illustration of the overall peak to peak current graph of different AEDs in comparison to the current AED as described.

FIG. 16 is an alternate simplified circuit diagram of a defibrillator.

### DETAILED DESCRIPTION OF PREFERRED EMBODIMENTS

Described is an automated external defibrillator (AED). The AED comprises two pads for placement on a patient, each pad comprising an energy storage system. The energy storage system comprises at least two energy storage blocks, a switching circuit and a shock generation circuit connected to the two pads, and a controller connected to the switching circuit and the shock generation circuit. The controller is configured to perform an electrical switching operation to provide a defibrillation shock in two phases, such that the voltage and a peak current in each of the two phases is substantially the same. Each energy storage block comprises at least one or more capacitors. In some aspects at least one of the energy storage blocks including two or more capacitors connected in series, and at least two energy storage blocks are connected in parallel so that the capacitor system includes capacitors connected both in series and in parallel with each other.

The inventors have developed an AED having a reduced form factor with a miniaturised but effective waveform. Thus the AED as described has a compact form factor with small pad footprints for component layout and packaging. At the same time, the pads are capable of acquiring ECG signals with high quality, and delivering defibrillation shocks with high efficacy.

The inventors have thus created and described a small form factor AED and a capacitor arrangement within AED that provides an optimal and/or efficient solution to create a waveform within the size constraints and within the limitations of current capacitor technology and regulatory standards.

The AED as described comprises the characteristics of:
a) low energy defibrillation, or
b) equal leading edge waveform, or
c) fully tilted waveform, or
d) independent capacitor banks for each phase, or
e) small form factor, or
f) any combination of two or more of (a) to (e).

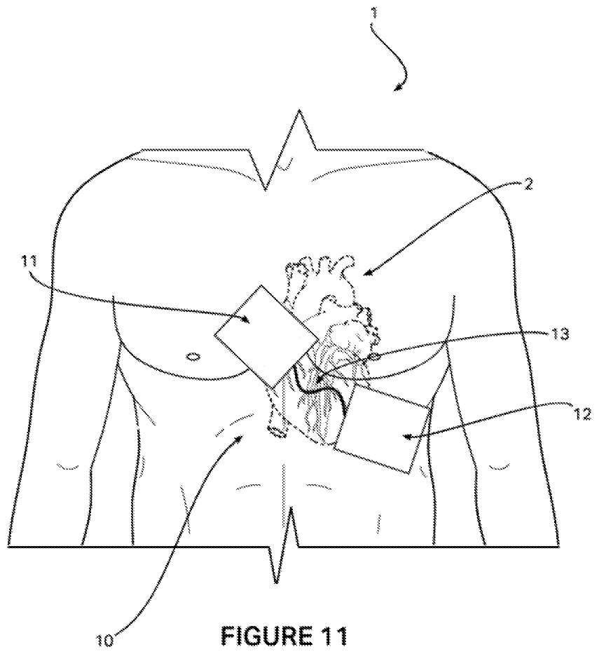

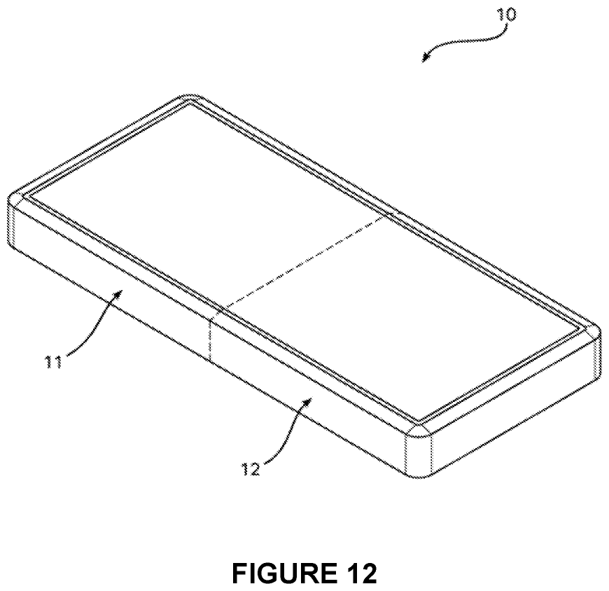

The AED (herein referred to as defibrillator) as shown in FIG. 12 according to an embodiment of the present disclosure may generally comprise two defibrillation pads 11 and 12. The pads 11 and 12 may initially be joined together but may be separable from one another and placed on a patient, for example in anterior-anterior locations for adults as illustrated in FIG. 11.

The AED 10 may have a compact device form factor with small pad footprints. The two pads 11 and 12 of the AED are configured to perform multiple functions of electrical measurement and stimulation of the patient's heart. A suitable compact AED is described in further detail in the present applicant's WO 2018/232450 which is hereby incorporated by reference in its entirety.

The defibrillator includes circuitry to enable distributing a pulse of energy to the patient for defibrillation through electrodes. To provide an energy pulse sufficient to cause defibrillation, the circuitry of a defibrillator may include one or more capacitors which can store then quickly discharge energy. The capacitors are charged for defibrillation by a power source. Particularly in the case of automatic external defibrillators (AEDs) the power source may be provided as part of the device, for example by a battery that is part of the AED. In other forms the power source may be provided external to the AED, for example through the battery of a mobile device.

An electronics module (not shown) may be packaged in the enclosures of each of the two pads. The electronics module may comprise a switching circuit and a shock generation circuit connected to the multiple electrode pairs The electronics module may further comprise a controller, such as one or more processors, connected to the switching circuit and the shock generation circuit.

The electronics module may further comprise other electronic components, such as one or more batteries, transformers, inductors which are also packaged the enclosures of one or both of the two pads. The electronic components of the AED 100 are described in further detail in the present applicant's WO 2018/232450 referred to above.

In an embodiment, an energy storage system of the present disclosure, comprises at least an energy storage system (also referred to as a capacitor system). The energy storage system comprises an energy storage bank (also referred to as a capacitor bank). The capacitor bank further comprises at least two energy storage blocks. In an embodiment, each of the energy storage blocks comprises at least one capacitor. In an alternate embodiment, each of the energy storage blocks according to the present disclosure may include at least three capacitors. The at least three capacitors may comprise two capacitors connected in series, and a third capacitor connected in parallel to one or both of the capacitors connected in series.

The capacitors of a defibrillator according to the disclosure may be of any suitable type. For example, the capacitors include one or more of film or power film capacitors, ceramic capacitors, supercapacitors, or electrolytic capacitors, or a combination thereof.

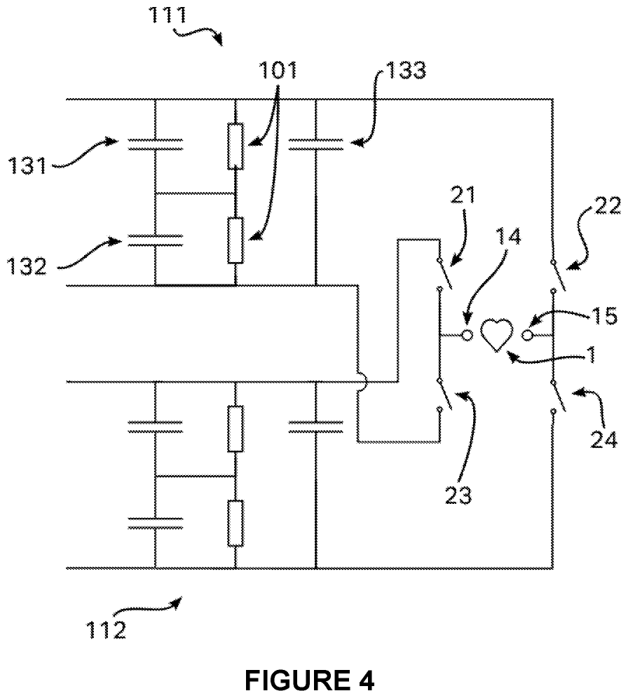

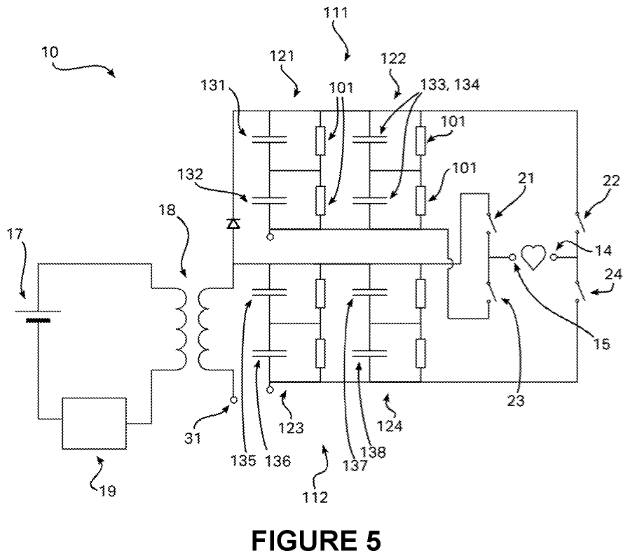

In an embodiment, as depicted in FIG. 4, the energy storage system comprises two balancing resistors 101. In alternative embodiments, the energy storage system may comprise a diode (as shown in FIG. 5), and/or an operational amplifier that is in addition to the balancing resistor, and is connected in series and/or parallel with the at least one capacitor in each of the energy storage blocks.

Accordingly, in a capacitor system comprising three or more capacitors, the capacitor system may comprise series connections between at least two capacitors, and parallel connections between at least two capacitors. i.e. for a three capacitor system that would comprise two capacitors connected in series, and another capacitor connected in parallel with one or both of the two series connected capacitors.

The capacitor system may include one or more banks of capacitors configured in this manner. In at least some configurations a given capacitor bank may be configured to store charge and discharge to provide energy for a single phase of a defibrillation waveform.

The parallel-connected elements of a capacitor system may be referred to as parallel energy storage blocks. Each of the parallel energy storage blocks may include one or more capacitors. Where it includes more than one capacitor, the capacitors of the energy storage block are connected in series.

Multiple energy storage blocks may together, as a or the capacitor bank of a capacitor system, store energy for discharging as a defibrillation shock.

As a capacitor bank of a capacitor system comprises two or more energy storage blocks, the energy storage blocks of a given capacitor bank may be charged and discharged together.

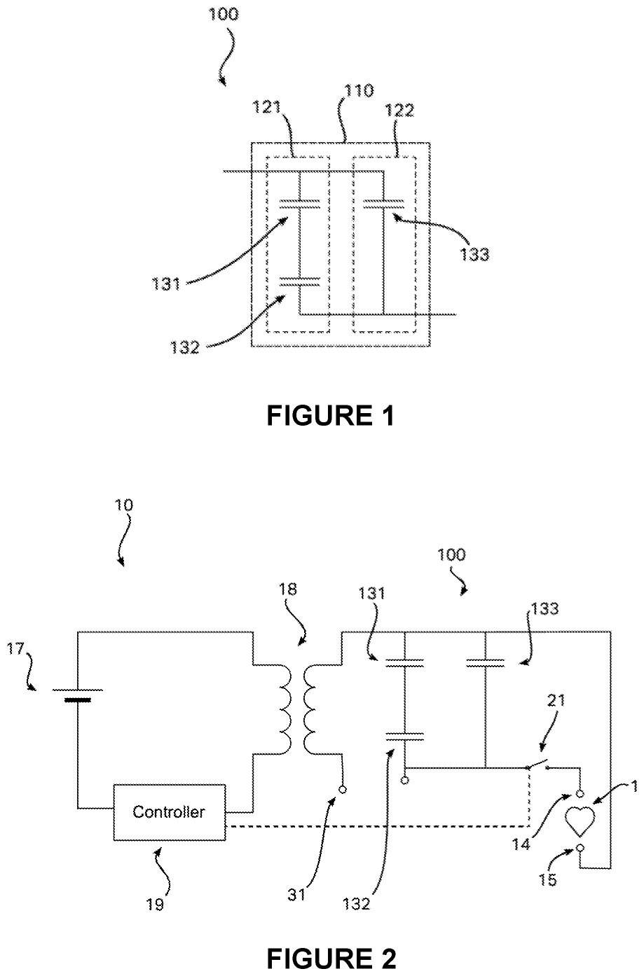

FIG. 1 shows an example of a capacitor system 100 for a defibrillator according to the disclosure. As seen in FIG. 1 the capacitors system 100 is made up of a single capacitor bank 110.

The energy storage system (or the capacitor system) 100 and the energy storage bank (capacitor bank) 110 are made up of a plurality of energy storage blocks, being a first energy storage block 121 and a second energy storage block 122.

The first energy storage block 121 has a first capacitor 131 and second capacitor 132 which are connected to each other in series. The second energy storage block 122 has a third capacitor 133. The first energy storage block 121 and second energy storage block 122 are connected to each other in parallel. The series and parallel connections between the capacitors which make up the capacitor system are fixed.

By configuring a capacitor system according to the disclosure an AED may be assembled using individual capacitors of a lower working voltage and/or rated capacitance than would be possible using either individual capacitors or multiple capacitors connected otherwise than in both series and parallel. The described configuration provides a balancing of the effects of series and parallel connections for both the overall working voltage and overall nominal capacitance.

When connected in series, a set of capacitors provide a total working voltage equal to the sum of the individual working voltages. However, the series connection also results in a reduction of the total capacitance, relative to the nominal capacitance of the individual capacitors. The total capacitance of capacitors in series is equal to the reciprocal of the sum of the reciprocals of the individual capacitor capacitances. This is described by the equation C_{T}=1/(1/C₁+1/C₂+1/C₃+ . . . ), where C_{T} is the total capacitance of the series set, C₁ is the capacitance of the first capacitor, C₂ is the capacitance of the second capacitor, and C₃ is the capacitance of the third capacitor.

The use of 30 μF to 60 μF dual capacitor choices may be chosen to provide optimal defibrillation in humans. However, beginning with these parameters there are limitations on producing that capacitance in a small form factor that stem from current limitations in capacitor technology.

In order to produce the required voltage and capacitance the arrangement needs to be optimised in relation to the size constraints of the electrical components used in the AED. The specific mathematical interaction between capacitors in series (c=(1/c₁+1/c₂+1/cₙ)⁻¹) and capacitors in parallel (c=C₁+C₂+ . . . +Cₙ) combined with the voltage constraints (summative in series, constant in parallel) makes it infeasible to use non-identical capacitors and provides diminishing returns when increasing the number of capacitors in series.

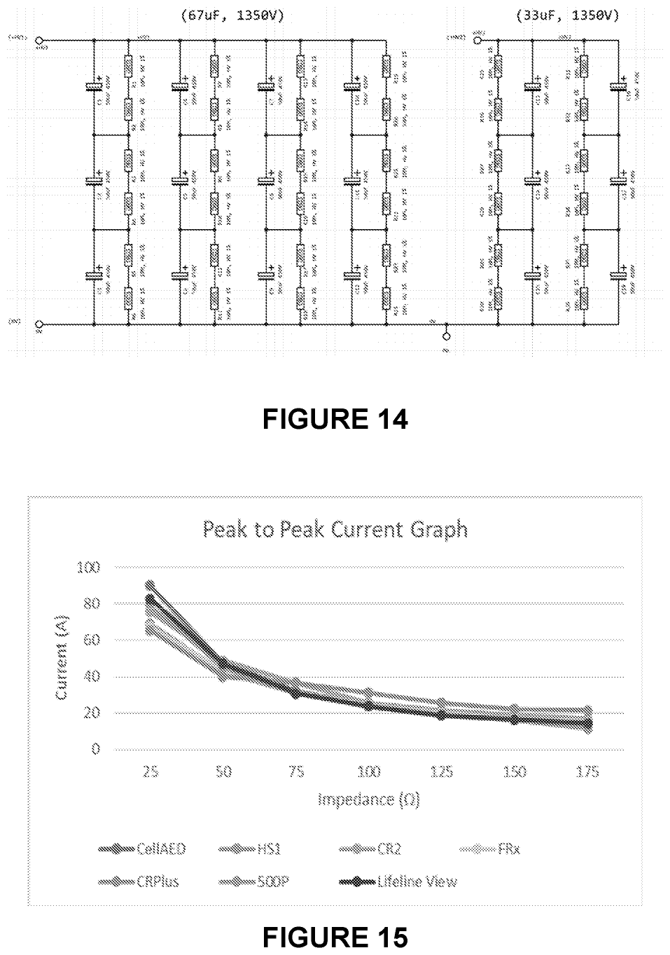

FIG. 14 depicts an exemplary embodiment of the capacitor configurations and the total capacitance of the AED.

When connected in parallel, capacitors have an effective capacitance equal to the sum of the individual capacitances, and a working voltage equal to the lowest working voltage of the parallel connected capacitors or sets of capacitors. Accordingly, when energy storage blocks having one or more series connected capacitors are connected in parallel, they will provide for a combined nominal capacitance which is the sum of the capacitances of each of the energy storage blocks.

A defibrillator may have a required working voltage and total capacitance to deliver a desired defibrillation shock to the patient. A given required working voltage and total nominal capacitance may be provided according to the disclosure by using a plurality of capacitors connected to include capacitors in series with each other and in parallel with each other.

The capacitor of an energy storage block may have a nominal capacitance of about 45, 46, 47, 48, 49, 50, 51, 52, 53, 54 or 55 μF, and useful ranges may be selected from any of these values (for example, about 35 to about 55, about 45 to about 53, about 45 to about 50, about 46 to about 55, about 46 to about 52, about 46 to about 50, about 47 to about 55, about 47 to about 53, about 47 to about 50, about 48 to about 55, about 48 to about 53, about 49 to about 55 or about 49 to about 50 μF).

The capacitor of an energy storage block may have a voltage of about 400, 425, 450, 475, 500, 525, 550, 570 or 600 V, and useful ranges may be selected from any of these values (for example, about 400 to about 600, about 400 to about 550, about 400 to about 450, about 425 to about 600, about 425 to about 500, about 425 to about 450, about 450 to about 600, about 450 to about 550 or about 450 to about 525 V).

A capacitor of an energy storage block may have a nominal capacitance of about 50 μF and a voltage of about 450 V. The nominal capacitance may be in the range of 6.8 μF to 60 μF and the voltage between 50 V to 600 V. The capacitance may have a requirement to achieve the desired waveform in the form factor available that leads to a particular configuration, whilst achieving the desired waveform.

In one embodiment, the first bank may be constructed of four parallel sets of three capacitors in series to a total rating of 67 μF and 1350 V. The second bank is two parallel sets of three capacitors in series to a total rating of 33 μF and 1350 V.

Due to the reduced form factor of the present biphasic defibrillator, one of the challenges of the present configuration is in relation to the amount of overall energy that can be generated by the defibrillator. However, in order to arrive at the optimal energy required for a successful defibrillation, the present AED is configured such that the optimal dosage of the defibrillation shock is derived from determining an adequate flow of defibrillation current (as seen in Table 1). Consequently, the present defibrillator achieves successful defibrillation of the heart with a lower energy dosage. This is not possible in conventional defibrillators where the focus is placed on determining optimal dosage.

Furthermore, pre-set energy levels vary across different AEDs on the market, ranging from 120 J (Zoll AED Pro/Plus) to 360 J (Primedic Heart Save). FIG. 15 depicts the overall peak-to-peak current graph of different AED's often used in the market, The graph maps the amount of current in Amperes on the X-axis vs. the amount of impedance in ohms on the Y-axis. As is evident, the current apparatus (identified as CellAED in red legend) achieves high peak currents and impedance values in comparison to the other devices commercially available, demonstrating the effectiveness of the defibrillator, in terms of achieving a successful defibrillation outcome in a patient.

The electronic components of the present AED may be configured to be operable in low power and low voltage modes. The interaction between each of the capacitors is largely similar to high power and voltage components used in a conventional defibrillator, that is otherwise orders of magnitude in size larger than the present AED.

An advantage of the present dual-bank capacitor arrangement is that it can maintain low power mode adaptability, yet emulate the higher powered (and larger) capacitor systems available in a conventional defibrillator. The low power mode adaptability can be achieved by performing multiple adjustments to the components of the charging circuit to account for the smaller battery. For example, adjustment to the circuitry may comprise (a) minimizing the current drawn by the circuit, especially the continuous “quiescent current”, (b) maintaining the voltage supplied at a constant level during discharge by using a regulating circuit, and/or (c) placing additional components such comparators and alternators.

As shown in FIG. 15, respective component values of the capacitor circuits are depicted. In some embodiments, the capacitors comprise all 50 μF, 450 V capacitors. The first bank may be constructed of four parallel sets of three capacitors in series to a total rating of 67 μF, 1350V. The second bank may comprise two parallel sets of three capacitors in series to a total rating of 33 μF, 1350V.

The peak currents and the voltage may be determined based on the transthoracic impedance of a particular patient. Typically reported values of impedance are about 50 Ohms. Tabulated data relating to peak current and voltage for adults and infants at various impedance values can be seen in Tables 1 and 2.

TABLE 1
Adult waveform properties.

| IMPEDANCE (Ω) | ENERGY (J) | V₁ (V) | PEAK I₁ (A) | T₁ (ms) | V₂ (V) | PEAK I₂ (A) | T₂ (ms) |
|---|---|---|---|---|---|---|---|
| 25 | 68 | 1050 | 42.0 | 5.2 | 1005 | 40.2 | 2.7 |
| 50 | 75 | 1150 | 23.0 | 9.7 | 1120 | 22.4 | 4.8 |
| 75 | 78 | 1183 | 15.8 | 14.2 | 1166 | 15.6 | 7.0 |
| 100 | 80 | 1204 | 12.0 | 18.8 | 1190 | 11.9 | 10.0 |
| 125 | 80 | 1212 | 9.7 | 20.1 | 1200 | 9.6 | 16.4 |
| 150 | 81 | 1224 | 8.2 | 20.0 | 1212 | 8.1 | 22.1 |
| 175 | 81 | 1235 | 7.1 | 20.0 | 1221 | 7.0 | 22.1 |

TABLE 2
Infant waveform properties.

| IMPEDANCE (Ω) | ENERGY (J) | V₁ (V) | PEAK I₁ (A) | T₁ (ms) | V₂ (V) | PEAK I₂ (A) | T₂ (ms) |
|---|---|---|---|---|---|---|---|
| 25 | 36 | 762 | 30.4 | 5.2 | 743 | 29.8 | 3.1 |
| 50 | 40 | 835 | 16.7 | 9.6 | 824 | 16.5 | 5.5 |
| 75 | 42 | 861 | 11.5 | 14.1 | 855 | 11.4 | 8.0 |
| 100 | 43 | 876 | 8.8 | 18.5 | 872 | 8.7 | 10.6 |
| 125 | 43 | 885 | 7.1 | 20.1 | 880 | 7.0 | 15.6 |
| 150 | 43 | 891 | 5.9 | 20.1 | 888 | 5.9 | 21.9 |
| 175 | 43 | 896 | 5.1 | 20.1 | 892 | 5.1 | 22.2 |

As is evident from Tables 1 and 2, the actual voltage achieved by the present defibrillator is lower than conventional defibrillators.

It will be appreciated that the energy storage system, and individual energy storage banks, may be configured according to the foregoing principles to provide a capacitor system to match a set of desired defibrillation waveform characteristics.

The shock waveform may be of the type having an equal leading edge in relation to the peak current in both the phases of the biphasic defibrillator. The waveform may be fully-tilted waveform enabling efficient energy application during the defibrillation shock, and ultimately, resulting in a lower overall energy.

The waveform may have a number of key parameters that go towards successful defibrillation comprising any one or more of:
Phase 1 peak current
Phase 1 duration.
Phase 2 peak current
Phase 2 duration

The duration of the first phase may have a minimum time (tp1) that must be achieved to ensure that the defibrillation shock reaches all the cells of the myocardium to achieve polarisation effect. The duration of the second phase is not mandated and is secondary to the first. The total duration of the shock is also regulated, as the shock is observed over a longer period will lead to an arrhythmia.

The magnitude of the shocks (peak currents) must be sufficient that they can polarise the heart cells in the first phase and depolarise in the second phase. The optimal peak currents observed are illustrated in the table 1 for adults and table 2 for infants.

It will also be appreciated that current and time are the factors that are most important to defibrillation efficacy, (not the energy). The energy of the shock may be calculated as being Energy=Current×Voltage×Time. Therefore, it is evident that the energy output is a by-product of the current and time, rather than the preferred outcome measure.

Referring to FIG. 15, a comparison of the claimed defibrillator in relation to a number of existing conventional biphasic defibrillators is shown. The results demonstrate that the peak-to-peak current is similar between all devices, highlighting the importance of current to defibrillation efficacy. Ultimately, the claimed defibrillation waveform has a comparable peak-to-peak current to other devices, however the fully-tilted waveform enables the present defibrillator to apply the energy more efficiently resulting in a lower overall energy.

Furthermore, the conventional biphasic waveform-based defibrillators use a truncated waveform that is partially tilted to achieve defibrillation shock in both phases. That is, part way through the shock switches are used to change the direction of current flow and hence change the phase of the shock, but the current is supplied from the same capacitor.

A distinction of the present design of the energy storage system is the use of the two independent energy storage banks. The controller may be configured to operate the switching and shock generation circuit such that, an electrical switching operation is performed, wherein each one of the energy storage banks (comprising the storage blocks) is used for a specific phase and achieve the fully tilted, equal leading edge waveform that cannot be created without that separation.

The use of two capacitor banks to achieve the biphasic waveform in the present configuration may reduce the complexity of the electrical circuitry and number of switches compared to single capacitor banks, making the present design more efficient. Thus, resulting in a small form factor of the defibrillator.

Furthermore, it is appreciated that the present day conventional biphasic waveforms use a truncated waveform that is partially tilted to achieve both phases. That is, part way through the shock, switches are used to change the direction of current flow, and hence change the phase of the shock. However, the current is supplied from the same capacitor.

In contrast, by using the two energy storage bank configuration of the present apparatus, the controller is configured to control the switching and shock generation circuit such that each of the energy storage banks (comprising the energy storage blocks) is used for a specific phase (i.e., either for a first phase or a second phase) of the two phases. For instance, the two energy storage banks are independent of each other for each of the two phases of the defibrillation shock.

In this embodiment, the switching circuit is configured to perform electrical switching operation such that one of the energy storage blocks is configured to charge, store and discharge to provide energy for one of the two phases, and the other of the energy storage blocks is configured to charge, store and discharge to provide energy for the other of the two phases of the defibrillation shock.

Additionally, the switching circuit is also configured to perform electrical switching operation such that the direction of the current flow is maintained to be the same during each of the two phases during the defibrillation shock. That is, in the present configuration, part way through the shock direction of current flow does not change. Consequently, the original direction that the current started in each of the two phases remains the same (or is maintained to be the same).

Therefore, the present configuration results in a fully tilted, equal leading edge waveform that cannot be created without the separation of the two phases (i.e., independent block for each phase).

The equal leading-edge waveform is generally in relation to the equal peak current between first phase and the second phase. In the present defibrillator, as the phases are handled with distinct energy storage banks, the defibrillator has equal current (peak current) and voltage parameters for each phase, even with the different capacitance values. In a conventional AED however, the voltage of the second phase is usually lower than the first as the capacitor(s) is discharged partially and loses voltage before the commencement of the second phase. This effect is not observed in the present description.

In this embodiment, the controller may be configured to perform an electrical switching operation to provide a defibrillation shock in two phases, such that the voltage and a peak current in each of the two phases is substantially the same.

The controller may be further configured to operate the shock generation and switching circuit to automatically perform electrical measurement and stimulation of the patient's heart switching between the two phases.

Furthermore, in the present configuration, by selecting the working voltages and nominal capacitances of the capacitors used, arranging the capacitors in series sets to provide a desired overall working voltage, and connecting these sets in parallel to increase the overall capacitance, the energy storage system according to the disclosure can match the desired working voltage and nominal capacitance of for the defibrillator.

The controller may be further configured to generate a predetermined dosage of current for defibrillation shock at a predetermined dosage of power. This determination is based on the pre-set/predetermined values for an adult or an infant as reported in the Tables 1 and 2, respectively.

Capacitors having lesser working voltages and/or rated capacitances may, for the same capacitor type, be of reduced size in at least one dimension. Capacitors with lower working voltages and/or rated capacitances may also, for the same capacitor type, be of a reduced cost. In particular, the sum of the cost of three or more of lesser rated capacitors may be less or even significantly less than the price of a lesser number of more highly rated capacitors.

Accordingly, an AED having a capacitor system configured according to the disclosure may be capable of providing one or both of a reduced total cost of the capacitor system and a capacitor system which can be configured into a shape that it smaller in at least one dimension than would otherwise be possible. The efficient design of an electrical circuit using low power components is the basis for reducing the form factor the present defibrillator as low power electrical components are smaller in size.

The use of two distinct capacitor banks to achieve the biphasic waveform may achieve a reduction in complexity and the number of switches compared to single capacitor banks, making the presently described design more practical and efficient.

Each pad of the present defibrillator may have a volume of about 100, 110, 120, 130, 140, 150, 160, 170, 180, 190 or 200 cm³, and useful ranges may be selected from any of these values (for example, about 100 to about 200, about 100 to about 180, about 100 to about 160, about 100 to about 150, about 110 to about 200. 110 to about 190, about 110 to about 170, about 110 to about 150, about 120 to about 200, about 120 to about 180, about 120 to about 150, about 130 to about 200, about 130 to about 180, about 130 to about 150, about 140 to about 200, about 140 to about 180, about 140 to about 160 or about 140 to about 150 cm³).

Each pad of the present defibrillator may have a surface area of about 50, 60, 70, 80, 90 or 100 cm², and useful ranges may be selected from any of these values (for example, about 50 to about 100, about 50 to about 80, about 50 to about 70, about 50 to about 60, about 60 to about 100, about 60 to about 80, about 60 to about 70 or about 50 to about 60 cm²).

For example, the volume of each of the pads may be about 9.7 cm×9.3 cm×1.7 cm to give a total volume of 153 cm³, and the surface area may be about 8.2 cm×8.6 cm to give a total surface area of 70. 5 cm².

The series and parallel connections between capacitors of a capacitor system, or at least of a given capacitor bank, may be fixed. In such a configuration the connections between the capacitors have non-switched connections between them. This may provide for simplicity and reliability compared to configurations where switches are included to change the series and parallel configurations of one or more capacitors.

Where the connections between the capacitors are fixed, they will be arranged in the same series and parallel relationships for both charging of the capacitors and their discharging. While increased charging efficiency may be gained through charging capacitors in parallel, the use of fixed connections in a capacitor system that includes capacitors connected in both series and parallel may provide a relatively reduced charging efficiency. However, any such reduced charging efficiency may be offset by the increased simplicity and reliability offered by fixed connections.

The capacitors may in at least some configurations be cylindrical or at least notionally cylindrical in shape, with a diameter and a longitudinal axis. The capacitors may be arranged in a planar configuration. Alternately the capacitors may have a mixed arrangement were some may be planar and some may be perpendicular.

FIG. 2 is a simplified circuit diagram of a defibrillator 10 such as an AED which includes the capacitor system 100 of FIG. 1.

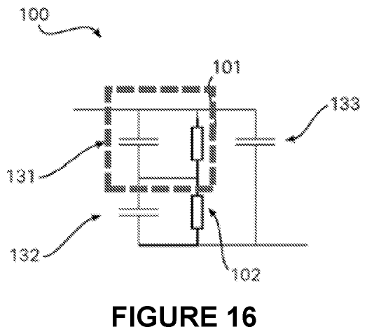

FIG. 16 is an alternate simplified circuit diagram of a defibrillator 10 such as an AED which includes the capacitor system 100 of FIG. 1. As depicted in this figure, the energy storage system comprises two energy storage banks comprising at least one capacitor 101 (as highlighted in red).

As seen in FIG. 2, the defibrillator 10 charges the capacitor system with energy from a battery 17. Before reaching the capacitor system 100 the voltage from the battery 17 is boosted through a transformer 18. The operation of the transformer 18 to charge the capacitor system 100 is controlled by a controller 19, which also controls the operation of a switch 21 to allow discharge of the capacitor system 100 to two electrodes 14 and 15 connected across the patient 1.

The series and parallel connections between the capacitors 131-133 of the capacitor system 100 are fixed so that they are the same during both the charging of the capacitors and their discharge to the patient.

The capacitors 131-133 of the capacitor system each have a nominal capacitance and working voltage. The capacitor system 100 will then have an overall nominal capacitance and working voltage based on the specifications of the individual capacitors.

While illustrated in a conceptual form in FIGS. 1 and 2, it will be appreciated that further capacitor systems may be constructed by application of the same principles to meet a desired defibrillation waveform of a defibrillator and particularly of an AED.

Furthermore, while the illustrated the circuit configuration of FIG. 2 may only provide for the delivery of only a single-phase defibrillation shock to a patient, the capacitor systems of the disclosure may be utilised in defibrillators that provide other types of defibrillation shocks. For example, biphasic or counter-shock defibrillation pulses are the common standard in AEDs.

Each phase of the biphasic shock may be provided by the each one of the two energy storage blocks by the incorporation of an H-bridge into the circuit of FIG. 2.

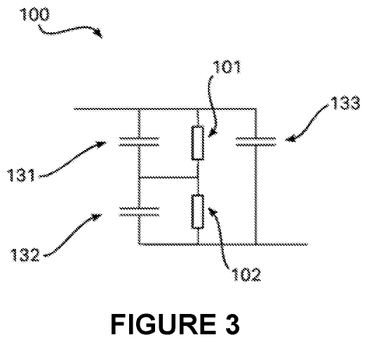

In other configurations separate capacitors may be utilised to provide each of the respective biphasic phases. Such configurations may also utilise an H-bridge to switch the polarity with which the charge is applied across the electrodes between the two phases.

Where capacitors are connected in series as part of an energy storage block one or more charge balancing resistors may be connected across each of the series capacitors to balance charges across each capacitor. For example, FIG. 3 shows the capacitor system 100 of FIG. 1 and FIG. 2, but where the first energy storage block 121 includes charge balancing resistors 101 and 102 associated respectively with the first capacitors 131 and second capacitor 132.

FIG. 4 is part of circuit diagram of a defibrillator 10 such as an AED showing a capacitor system 100 and electrodes 14 and 15. The capacitor system 100 has a first capacitor bank 111 and a second capacitor bank 112. The first capacitor bank 111 is for delivering the first phase of a biphasic defibrillation shock. The second capacitor bank 112 is for delivering the second phase of a biphasic defibrillation shock.

The first capacitor bank 111 may be made up of a first energy storage block 121 and second energy storage block 122. The first energy storage block 121 may have capacitors 131 and 132 connected in series. The second energy storage block 122 may comprise a single capacitor 133.

The second capacitor bank 112 may comprise a third energy storage block which may have capacitors 134 and 135 connected in series, and a fourth energy storage block which may have a single capacitor 136.

The energy storage blocks 121 and 122 of the first capacitor bank 111 may be connected in parallel. Similarly, the energy storage blocks 123 and 124 of the second capacitor bank 112 may be connected in parallel.

Both of the first energy storage block 121 and third energy storage block 123 may comprise balancing resistors 101a-101d connected across each of the respective series connected capacitors.

The two capacitor banks 111 and 112 of FIG. 4 are connected to respective electrodes 14 and 15 by four switches 21-24 which provide the function of an H-bridge. By selective operation of the switches 21-24, such as by a controller 19, the capacitor banks 111 and 112 of the capacitor system 100 may be successively discharged to provide a biphasic defibrillation shock.

A switch may be of any commonly available form, such as a transistor (for example FET or BJT switch), or a relay.

The nominal capacitance and working voltages of the capacitor banks 111 and 112 may be the same or may be different, as desired to provide a desired biphasic defibrillation shock.

While a capacitor system 100 or a capacitor bank may comprise only three capacitors, in at least some configurations the capacitor system or capacitor bank may include more than three capacitors connected in combinations of series and parallel.

The capacitors of a capacitor system according to the disclosure may be selected in order to provide a desired working voltage and/or nominal capacitance to one or each of individual energy storage blocks, each capacitor bank, and the capacitor system as a whole. For example, the number of capacitors in each energy storage block and their individual working voltages and nominal capacitances may be selected to provide, as a result of their series and parallel combinations, a particular working voltage and/or nominal capacitance for a given energy storage block, for a particular one of the capacitor banks, or for the entire capacitor system as a whole.

In some configurations the capacitors of a capacitor bank, or even of a whole capacitor system, may be selected to have the same working voltages.

While different energy storage blocks of the same capacitor bank may include different numbers of capacitors, to maximise efficiency it may be preferable that the total working voltage of each energy storage block is the same. Otherwise, in order to not exceed the total working voltage of the lowest rated energy storage block, other blocks would be charged to beneath their working voltage.

Energy storage blocks with the same total working voltage may be provided by series combinations of either the same or different capacitors.

In at least some configurations the capacitors of a capacitor bank or of a whole capacitor system may be selected to at least have one physical dimension in common. For example, in the case of cylindrical capacitors, the specifications of the capacitors such as nominal capacitance and working voltage may be the same or different, but at least one of the diameters of the capacitors and their lengths may be the same or substantially the same.

By employing capacitors having a shared dimension a corresponding dimension of the defibrillator or defibrillator part or parts that include the capacitors may be reduced.

FIG. 5 is a simplified circuit diagram of a defibrillator with a capacitor system 100 made up of a first capacitor bank 111 and second capacitor bank 112. Each capacitor bank 111 and 112 is made up of two energy storage blocks 121-122 and 123-124, each having a respective two of the capacitors 131-138 connected in series. Balancing resistors 101 are connected across each capacitor 131-138.

As seen in FIG. 5 the capacitor system side of the transformer 18 is shown connected to a zero-voltage node 31.

In the example of FIG. 5 the energy storage blocks 121 and 122 of the first capacitor bank 111 may have the same working voltage. This may be provided by either a) respective pairs of the capacitors of each of the energy storage blocks 121 and 122 having the same working voltage, b) each capacitor having a different working voltage (for example capacitor 131 having a working voltage of 200 V and capacitor 132 having a working voltage of 300 V for a first energy storage block 121 working voltage of 500 V, and capacitor 133 having a working voltage of 150 V, and capacitor 134 having a working voltage of 350 V for a second energy storage block 122 working voltage of 500 V), or c) by each of the capacitors 131-134 having the same working voltage.

An extension of the same considerations applies to a capacitor bank having more than two capacitors in series in one or each energy storage block.

As seen in FIG. 5, the capacitors of each of the capacitor banks 111 and 112 have fixed non-switched relationships to each other, so the capacitors will be charged and discharged in the same configuration of series and parallel relationships.

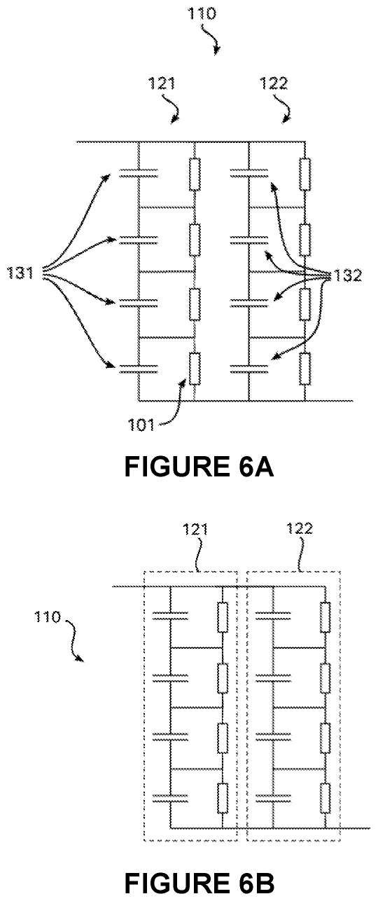

FIGS. 6A and 6B are circuit diagrams of another example configuration of a capacitor bank 110. In some configurations the capacitor bank 110 may form the capacitor system 100. In other configurations a capacitor system 100 may include two or more of the capacitor banks 110.

The energy storage bank 110 of FIG. 6A has a first energy storage block 121 and second energy storage block 122, as illustrated in FIG. 6B. Each energy storage blocks 121 and 122 includes four capacitors 131-134 and 135-138. Balancing resistors 101 are connected across each capacitor.

In the configuration of FIGS. 6A and 6B the capacitors of each energy storage block may be selected to have at least similar, or preferably the same, total working voltage. The total nominal capacitance of each energy storage block 121 and 122 may be the same or may be different.

The capacitors 131 and 132 may comprise identical sets of capacitors.

The capacitors 131 and 132 may each have the same working voltage and/or nominal capacitance.

The capacitors 131 and 132 may each have at least one dimension in common, for example where the capacitors are of a cylindrical format, the capacitors may each have the same or at least approximately the same diameter or length.

While the capacitor banks of a capacitor system may be made up of a corresponding number of energy storage blocks and/or capacitors, in at least some configurations different capacitor banks of a capacitor system may be made up of one or both of different numbers of energy storage blocks and different numbers of capacitors. Capacitor banks may also either have the same or different total working voltages and nominal capacitances.

Where separate capacitor banks are used to discharge each phase of a defibrillation shock, the configuration of each capacitor bank may be customised to provide the desired characteristics of each phase.

For example, in at least some configurations it may be desirable to deliver a greater defibrillation energy in a first defibrillation pulse than in a second defibrillation pulse.

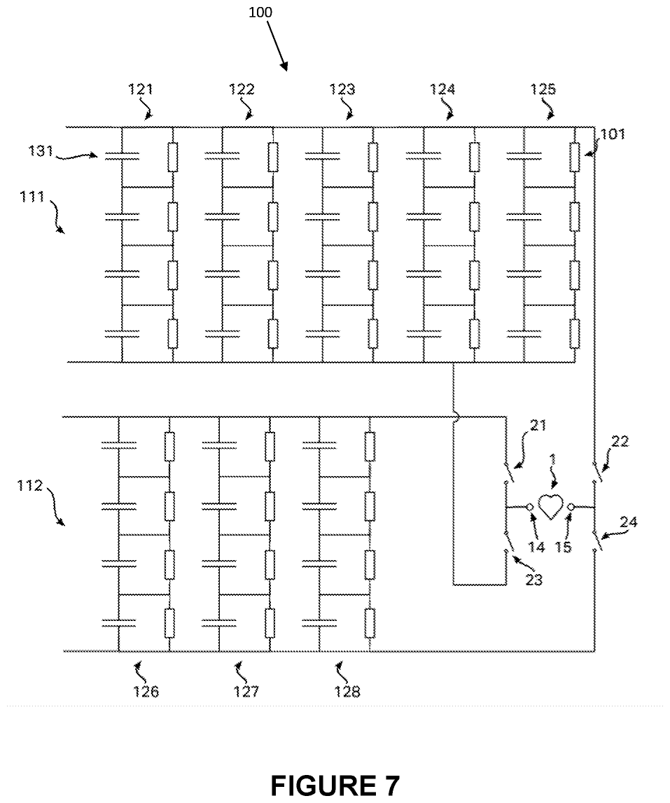

FIG. 7 is a partial circuit diagram of a defibrillator showing a capacitor system 100 having a first capacitor bank 111 and second capacitor bank 112, and an H-bridge formed by the switches 21-24 to selectively connect the capacitor banks 111 and 112 to the electrodes 14 and 15. A patient 1 is illustrated between the two electrodes 114 and 115.

The first capacitor bank 111 is made up of five energy storage blocks 121-125, each of which include four series connected capacitors 131, with a balancing resistor 101 connected across each.

The second capacitor bank is made up of three energy storage blocks 126-128, each of which similarly include four series connected capacitors 132 with a balancing resistor 101 connected across each.

The capacitors of the first capacitor bank may be capacitors of the same working voltage and nominal capacitance ratings. They may alternatively include sets of differently rated capacitors. They may further alternatively each be differently rated from one another. The same applies to the capacitors of the second capacitor bank.

In addition, or alternatively to any particular configuration of the ratings of the capacitors, the capacitors of one or both of the capacitor banks may share at least one physical dimension. For example, where the capacitors are cylindrical, they may have a common diameter and/or length.

In a configuration where each of the capacitors 131 and 132 have a common working voltage and/or nominal capacitance, or at least each of the energy storage blocks 121-128 have a common overall working voltage and/or nominal capacitance, the second capacitor bank 122 will have a lower total energy capacity than the first capacitor bank 121.

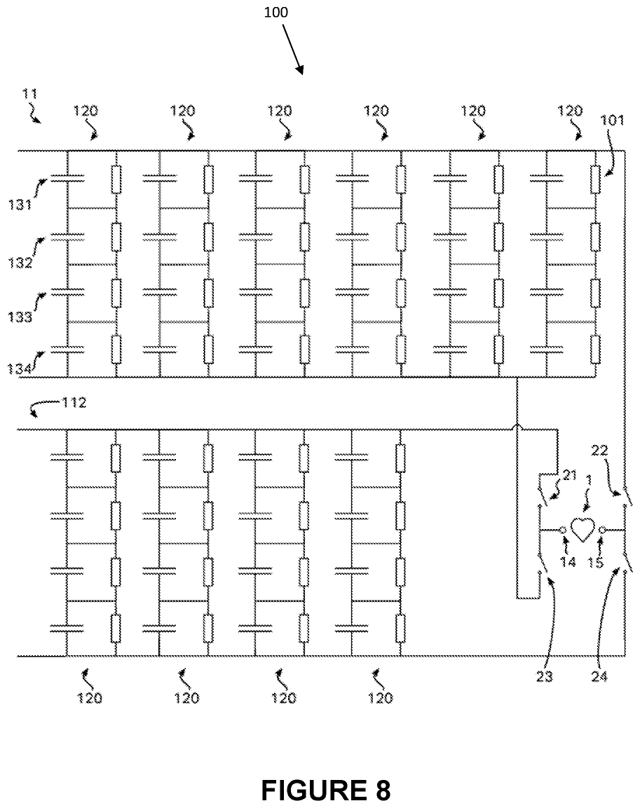

FIG. 8 is a partial circuit diagram of a defibrillator showing another configuration of a capacitor system 100. The capacitor system 100 of FIG. 8 has a first capacitor bank 111 and second capacitor bank 112. The capacitor banks 111 and 112 are connected to electrodes 14 and 15 by way of four switches 21-24 which act as an H-bridge. The capacitor banks 111 and 112 are each made up of energy storage blocks 120 each of which have four capacitors 131-134 connected in series. The first capacitor bank 111 is made up of six energy storage blocks 120, while the second capacitor bank 112 is made up of four energy storage blocks 120. Each capacitor of each energy storage block 120 includes a balancing resistor 101 connected across it to balance charges between the capacitors 131-134 of the respective energy storage block 120.

In the configuration of FIG. 8 where each energy storage block 120 has the same total working voltage and nominal capacitance, the first capacitor bank 121 may have 50% more energy storage capacity than the second capacitor bank 122.

While the foregoing provides examples of capacitor systems and capacitor banks of capacitor systems, it will be appreciated that many other configurations of capacitor systems and their capacitor banks may be arrived at through application of the foregoing principles of combining with fixed connections capacitors to include capacitors connected in both series and parallel. In particular, capacitors may be selected and arranged within energy storage blocks to provide capacitor banks, where desired, and a capacitor system as a whole, having any desired working voltage and/or nominal capacitance.

For example, according to one configuration of an AED the capacitor system may be desired to supply first phase of a biphasic defibrillation shock at about 2 kV, with a total phase energy of about 20 J. This requires a first phase supply with a nominal capacitance of about 10.2 μF.

Such a configuration may be provided by, for example, a first capacitor bank having six energy storage blocks, each comprising four series connected capacitors which each have a working voltage of 450 V and a nominal capacitance of 50 μF.

To further the example, in this configuration the second phase may be required to supply the second phase of the biphasic defibrillation shock at about 2 kV with a total phase energy of about 16.5 J. This requires a second phase supply with a nominal capacitance of about 6.8 μF.

These specifications may be provided by, for example, a second capacitor bank having four energy storage blocks, each comprising four series connected capacitors which each have a working voltage of 450 V and a nominal capacitance of 50 μF.

In this example, each capacitor bank is made up of capacitors with the same specifications, and the specifications of the capacitors are the same between the two capacitor banks. Such a configuration may allow for the use of physically identical capacitors, such as may have identical dimensions. For example, where the capacitors are cylindrical capacitors, each of the capacitors of each capacitor bank and the capacitor system as a whole may have the same diameter and length. This may allow for increased efficiency in the dimensions of the AED or AED part or parts within which the capacitors are provided.

In another example, according to another configuration of an AED, the AED may be desired to supply a total biphasic defibrillation energy of about 100 J. This energy may be distributed unequally between the two phases, for example with about 67 J to be delivered by the first phase and about 33 J to be delivered by the second phase. For the purposes of this example, the defibrillation phases may each need to be delivered at about 1.5 kV.

These specifications require a working capacitance of about 60 μF for the first phase and nominal capacitance of about 30 μF for the second phase.

For the first phase this may be provided by, for example, a first capacitor bank having four energy storage blocks, each with three series-connected capacitors that each have a working voltage of 450 V and a nominal capacitance of 67 μF and 1350 V. For the second phase the specified nominal capacitance may be provided by, for example, a second capacitor bank having two energy storage blocks, each with three series-connected capacitors that each have a working voltage of 450 V and a nominal capacitance of 33 μF and 1350 V.

Such characteristics may include, in the case of biphasic defibrillation, one or more of the peak voltages of one or both phases and the defibrillation energy of each respective phase or the total of both phases.

In at least some embodiments electrolytic capacitors may be utilised in a capacitor system due to factors such as the relatively high capacitance per unit volume that they can provide.

More particularly but not solely, in some configurations roll-type electrolytic capacitors may be utilised.

While in some of the foregoing examples the capacitor banks individually and capacitor system as a whole are made up of identically rated capacitors, it will be appreciated that the same total working voltage and phase energy requirements may be provided by any number of other combinations of differently rated capacitors, when combined in parallel connected energy storage blocks, at least one of which includes capacitors connected in series.

In at least some preferred configurations however the total working voltage of each energy storage block of a given capacitor bank will be approximately equal, and at least one physical dimension of each of the capacitors of a given capacitor bank will be approximately equal.

While in at least some of the foregoing examples the working voltages of both the first capacitor bank and second capacitor bank are equal, in some configurations the capacitor banks may have unequal working voltages, in addition to or instead of different total nominal capacitances, as needed to provide the desired defibrillation phase waveform characteristics.

As previously described, in at least some configurations the capacitors of a capacitor bank or the whole capacitor system may share only one dimension, such as in the case of a cylindrical capacitor either diameter or length.

The disclosure also provides for a defibrillator, and particularly an AED, which has one or more particular spatial arrangements of the capacitors that are provided as part of the AED.

Many factors may influence the availability of AEDs when and where they are needed for treating sudden cardiac arrests. Price may be a significant factor in determining the widespread availability of AEDs, especially in less wealthy communities and regions. The weight and/or physical size of the device may also be a significant factor in determining when and where AEDs are available. The weight of an AED may place practical limits on whether people will be willing to carry them on their person or keep one close at hand in case of an emergency. Similarly, the physical dimensions of an AED may practically limit its uptake. For example, a large device may not be practicable for a person to carry with them, or convenient to keep within reach in day-to-day situations such as in a workplace, in a vehicle, or in the home.

According at least some configurations of the disclosure, a defibrillator such as an AED may be provided having a reduced size in at least one dimension.

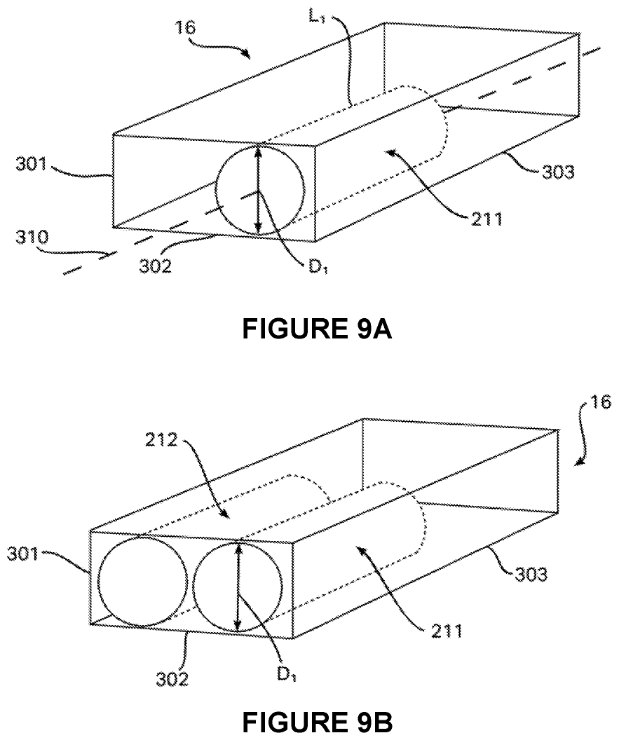

FIG. 9A is a view of a housing 16. In various configurations the housing 16 may be the housing of an integrated single-piece defibrillator, such as an AED, the housing of an electrode pad module where the components of the AED are associated with the pads, or the housing of a defibrillator base unit, to which each of the electrode pads are connected.

Within the housing 16 of FIG. 9A a capacitor 211 is shown. The housing 16 has a height 301, width 302, and depth 303. The capacitor 211 is of a cylindrical format with a diameter D₁ and a length L₁. The capacitor 221 has a cylindrical axis 310.

At least one or potentially all three dimensions of the housing 16 may be limited by the diameter D₁ and a length L₁ of the capacitor 221. For example, as seen in FIG. 9A, the height 301 of the housing 16 is limited by the diameter D₁ of the capacitor 211.

FIG. 9B shows another example of a housing 16 such as described in relation to FIG. 3A, but where the housing includes two capacitors 211 and 212, each with a diameter D₁ and a length L₁ (not shown). In this configuration both the height 301 and width 302 of the housing 16 are limited by the diameter D₁ of the two capacitors 211 and 212.

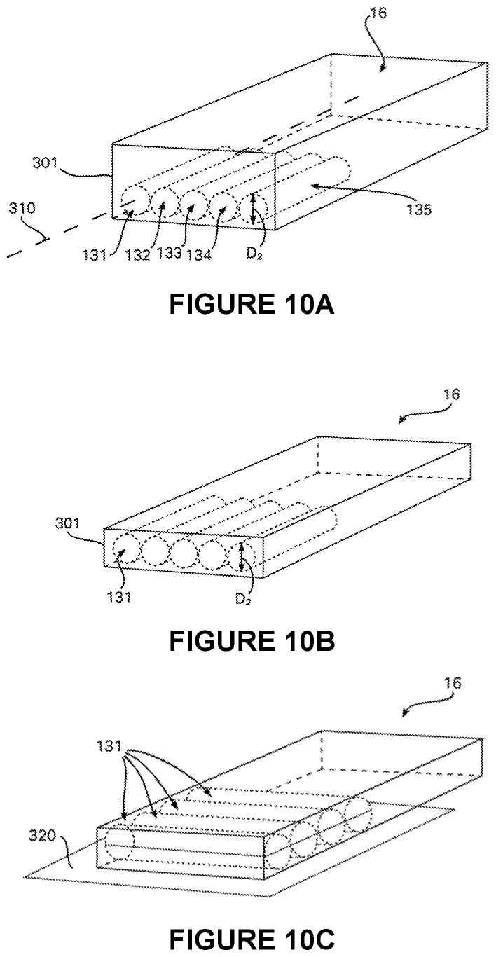

FIG. 10A illustrates another housing 16, having a plurality of capacitors 131-135, each having a diameter D₂ and a length L₂ (not shown). The plurality of capacitors 131-135 may be capacitors of a capacitor system of the disclosure.

The diameter D₂ of the capacitors 131-135 are less than that of the capacitors 211 and 212 of FIGS. 9A and 9B. Accordingly, as illustrated in FIG. 10B, the height 301 of the housing 16 of FIG. 4A may be reduced relative to the housing of, for example, FIGS. 9A or 9B, or 10A, providing a defibrillator or part of a defibrillator which is of relatively reduced size in one dimension. This reduction may additionally allow a reduction in the overall volume of the housing 16.

Each of the capacitors 131-135 have a cylindrical axis 310 as illustrated on the capacitor 131 of FIG. 10A. As seen in FIGS. 10A and 10B the cylindrical axes of each of the capacitors 131-135 are oriented parallel to the length 303 of the housing 16. Alternatively, the capacitors have a longitudinal axis (not shown)

As seen in FIGS. 10A-C the capacitors 131-135 are arranged so that their cylindrical axes are parallel.

FIG. 10C illustrates a notional plane 320. In the configuration illustrated in FIG. 10C, the capacitors 131-134 are arranged so their cylindrical axes pass through the plane 320. The cylindrical axes of the capacitors may be said in this configuration to be co-planar with the plane 320.

In some configurations the housing 16 may include or be associated with an electrode pad. The electrode pad may have a planar form. In such configurations, and where the cylindrical axes of the capacitors 131 are located within the plane 320, the plane 320 may also be parallel with the plane of the electrode pad.

As seen in FIG. 10C the capacitors 131-134 are oriented so that their cylindrical axes are perpendicular to the length 303 of the housing 16.

A capacitor system of the disclosure may be included in a defibrillator, for example an AED. An example form of an AED 10 is illustrated in FIG. 11 deployed for use on the torso of a patient 1. The patient's heart 2 is shown in dashed lines within their chest.

The AED 10 of FIG. 11 has a first pad module 11 and a second pad module 12 with a connecting wire 13 connecting the two modules. Electrodes or electrode pads 14 and 15 are located on the patient-facing sides of the pad modules 11 and 12, to transmit the defibrillation shock to the patient. The required operational components of the AED 10, particularly the capacitor system 100 may be located as part of one or between both of the pad modules 11 and 12.

In other forms, such as where the AED 10 has a base module which connects to the two electrode pads 14 and 15, some or all the other operational components of the AED, including particularly the capacitor system, may be located in the base unit.

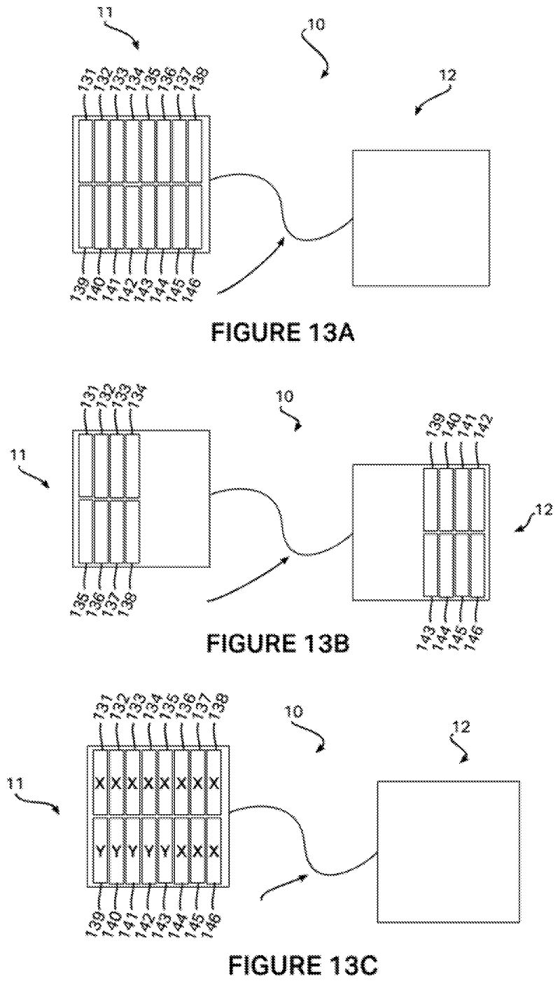

FIGS. 13A-C are plan view schematic illustrations are different configurations of a capacitor system 100 within an AED 10 which has two pad modules 11 and 12.

In addition, or as an alternative to being arranged axially parallel, two or more of the capacitors of a capacitor system may be arranged co-axially. This is illustrated in FIG. 13A, for example by the capacitors 131 and 139 are parallel.

In FIG. 13A the capacitors 131-146 make up the capacitor system 100. The capacitors 131-146 are provided as part of the first pad module 11. This configuration is also illustrated in FIG. 13C.

In FIG. 13B the capacitors 131-146 that make up the capacitor system 100 are distributed between the first pad module 11 and the second pad module 12.

Where a capacitor system 100 includes multiple capacitor banks, the physical arrangement of the capacitors in the capacitor system may, at least in part, reflect the allocation of the capacitors into banks.

For example, in the configuration of FIG. 13A the capacitors 131-138 may belong to a first capacitor bank, while the capacitors 139-146 may belong to a second capacitor bank. In such a configuration the capacitors of each bank may be co-axially aligned with a corresponding capacitor of the other bank. In this configuration the axes of the capacitors may further be located in common plane.

In another example, in the configuration of FIG. 13A the capacitors 131-134 and 139-142 may belong to a first capacitor bank, while the capacitors 135-138 and 143-146 may belong to a second capacitor bank. In such a configuration the capacitors of each phase may be co-axially aligned with another capacitor of the same phase.

FIG. 13C illustrates a configuration where there are two capacitor banks 111 and 112 of an unequal number of capacitors. The capacitors of the capacitors banks 111 and 112 are denoted respectively by “X” and “Y”. As seen in FIG. 13C, the capacitors are arranged in two rows, so that a capacitor of each row is co-axially aligned with corresponding capacitor of the other row.

The capacitors of a capacitor system may be arranged in a defibrillator, and particularly in an AED, in any desired number of columns and rows.

A capacitor system may include multiple layers of capacitors, where the layers are arranged in a direction into or out of the page of the schematics of FIGS. 13A-C.

However, in at least some preferred configurations the capacitors of a capacitor system according to the disclosure may be arranged in a single layer.

In some configurations a capacitor system having capacitors which are arranged so the longitudinal axes of each capacitor are located within a common plane may have the capacitors connected in fixed combinations of both series and parallel as described for example in relation to FIGS. 1-8.

In other configurations however a capacitor system may have capacitors which are arranged so the longitudinal axes of each capacitor are located within a common plane, but with the capacitors connected together other than in fixed combinations of series and parallel.

A capacitor system according to the disclosure may be charged and discharged in the operation of a defibrillator such as an AED. Where a capacitor system includes more than one bank of capacitors, the banks may be charged from a power source either sequentially or at the same time. Where a capacitor system includes more than one bank of capacitors, the banks may be discharged all at the same time, one at a time, or in different combinations as desired to provide a desired defibrillation waveform.

A capacitor system according to the disclosure may be manufactured by first fixedly connecting a plurality of capacitors in series to form an energy storage block. A plurality of these energy storage blocks may be formed. The energy storage blocks may be fixedly connected together in parallel to form the capacitor system.

The steps of assembling the energy storage blocks and joining them together in parallel may be completed in either order or at the same time.

While generally described in relation to AEDs, capacitor systems according to the disclosure may also be utilised as part of other types of defibrillators such as implantable cardioverter-defibrillators (ICDs), extra-cardiac implantable defibrillators (EIDs), or other forms of non-AED external heart defibrillators (EHDs).

In an embodiment, the voltage ratio between both the banks may be equal. In alternate embodiments, the voltage ratio may be different. Similarly, the energy storage ratio in certain embodiments is about 2:1. Depending on the configuration and requirements, this ratio may change.

A method of operating an AED having two pads for placement on a patient comprises. (i) performing multiple functions of electrical measurement and stimulation of the patient's heart, and (ii) operating a controller to perform an electrical switching operation to provide a defibrillation shock in two phases, wherein a voltage and a peak current in each of the two phases is substantially the same.

In an embodiment, the peak current and voltage in the first phase are maintained until a first time interval tp1 in which a polarisation effect is observed in the patient, The first time interval is the time taken for the defibrillation shock to reach all cells of myocardium of the patient. The multiple functions of electrical measurement and stimulation of the patient's heart performed by the one or more electrodes in multiple directions comprise:
(i) measuring cardiac electrical signals to detect locations of the two pads;
(ii) measuring ECG signals to detect shockable cardiac rhythms; and
(iii) delivering doses of defibrillation shocks by the two pads based on their detected locations when shockable cardiac rhythms are detected.

Where in the foregoing description reference has been made to elements or integers having known equivalents, then such equivalents are included as if they were individually set forth.

Although embodiments have been described with reference to a number of illustrative embodiments thereof, it will be understood by those skilled in the art that various changes in form and details may be made therein without departing from the spirit and scope of the invention as defined by the appended claims. Therefore, the preferred embodiments should be considered in a descriptive sense only and not for purposes of limitation, and also the technical scope of the invention is not limited to the embodiments. Furthermore, the present invention is defined not by the detailed description of the invention but by the appended claims, and all differences within the scope will be construed as being comprised in the present disclosure.

Many modifications will be apparent to those skilled in the art without departing from the scope of the present invention as herein described with reference to the accompanying drawings.

The invention claimed is:

1. An automated external defibrillator (AED) comprising
two pads for placement on a patient, each pad comprising an energy storage system; the energy storage system comprising at least two energy storage blocks,
a switching circuit and a shock generation circuit connected to the two pads, and
a controller connected to the switching circuit and the shock generation circuit, the controller configured to perform an electrical switching operation to provide a defibrillation shock in two phases, such that the voltage and a peak current in each of the two phases is substantially the same.

2. An AED of claim 1, wherein the at least two energy storage blocks are independent of each other for each of the two phases of the defibrillation shock.

3. The AED of claim 1, wherein the at least two energy storage blocks are connected in parallel, each energy storage block comprising at least one capacitor and at least one of the energy storage blocks comprising two or more capacitors in series.

4. The AED of claim 3, wherein the series and parallel arrangement of the capacitors are the same during both charging of the energy storage blocks and discharging of the energy storage blocks, to provide a defibrillation shock.

5. The AED of claim 1, wherein each pad has a volume of about 100 cm³ to 200 cm³, and a surface area of about 50 cm² to 100 cm².

6. The AED of claim 1, wherein the controller is further configured to produce an equal leading edge waveform for each of the two phases.

7. The AED of claim 1, wherein the controller is further configured to generate a predetermined dosage of current for defibrillation shock at a predetermined dosage of power.

8. The AED of claim 1, wherein the controller is further configured to maintain peak current in each phase such that the polarisation effect is observed in the first phase and a depolarisation effect is achieved in the second phase.

9. The AED of claim 1, wherein the controller is further configured to produce a fully tilted waveform for each of the two phases.

10. The AED of claim 1, wherein the switching circuit is configured to perform electrical switching operation such that one of the energy storage blocks is configured to charge, store and discharge to provide energy for one of the two phases, and the other of the energy storage blocks is configured to charge, store and discharge to provide energy for the other of the two phases of the defibrillation shock.

11. The AED of claim 10, wherein the switching circuit is configured to perform electrical switching operation such that the direction of the current flow is maintained during each of the two phases during the defibrillation shock.

12. The AED of 1, wherein each of the capacitors of at least one of the energy storage blocks have the same or substantially the same nominal capacitance and working voltage.

13. The AED of claim 1, wherein each energy storage block further comprises any one or more of a balancing resistor, a diode, or an operational amplifier connected in series and/or parallel connection with the at least one capacitor in each of the energy storage blocks.

14. The AED of claim 1, wherein the AED further comprises any one or more of a transformer, electrical switch, battery and an inductor, and wherein, each of the transformer, electrical switch, battery and the inductor are configured to be operable in a low voltage or a low power mode.

15. The AED of claim 1, wherein the shock generation circuit comprises a charging circuit and/or a discharging circuit configured to charge and/or discharge the one or more capacitors of the energy storage blocks.

16. The AED of claim 1, wherein the controller is configured to operate the shock generation and switching circuit to automatically perform electrical measurement and stimulation of the patient's heart switching between the two phases.

17. The AED of claim 1, wherein each of the two pads comprises one or more electrodes, and wherein the at least one electrode of each pads is configured to carry out at least one of an electrical measurement and stimulation of the patient's heart.

18. The AED of claim 1, wherein the energy storage system comprises at least six energy storage blocks, and wherein four energy storage blocks are configured to charge, store and discharge to provide energy for the first of the two phases of the defibrillation shock, and wherein the other two energy storage blocks are configured to charge, store and discharge to provide energy for the second of the two phases of the defibrillation shock.

19. The AED of claim 1, wherein the at least two energy storage blocks are connected are connected in parallel, each energy storage block comprising at least one capacitor.

20. A method of operating an AED having two pads for placement on a patient, the method comprising:
performing multiple functions of electrical measurement and stimulation of the patient's heart, and
operating a controller to perform an electrical switching operation to provide a defibrillation shock in two phases, wherein a voltage and a peak current in each of the two phases is substantially the same.

21. The method of claim 20, wherein the peak current and voltage in the first of the two phases of the defibrillation shock are maintained until a first time interval tp1 in which a polarisation effect is observed in the patient.

22. The method of claim 21, wherein the first time interval is the time taken for the defibrillation shock to reach all cells of myocardium of the patient.

23. The method of claim 20, wherein the multiple functions of electrical measurement and stimulation of the patient's heart performed by the one or more electrodes in multiple directions comprise:
measuring cardiac electrical signals to detect locations of the two pads;
measuring ECG signals to detect shockable cardiac rhythms; and
delivering doses of defibrillation shocks by the two pads based on their detected locations when shockable cardiac rhythms are detected.

24. The method of claim 20, wherein the measured cardiac electrical signals used to detect locations of the two pads comprise voltage, current, impedance, or any combination thereof.
